(19) United States
(12) Patent Application Publication
(10) Pub. No.: US 2018/0028344 A1
(43) Pub. Date: Feb. 1, 2018

(54) CERVICAL COLLAR

(71) Applicant: Bo Bae KIM, Seoul (KR)

(72) Inventor: Bo Bae KIM, Seoul (KR)

(21) Appl. No.: 15/664,440

(22) Filed: Jul. 31, 2017

(30) Foreign Application Priority Data
Aug. 1, 2016 (KR) 10-2016-0097929

(51) Int. Cl. A61F 5/055

(52) U.S. Cl. CPC A61F 5/055

(57) ABSTRACT

Disclosed is a cervical collar, in which the cervical collar is configured such that the outer shell and the inner shell thereof are detachably separable. Thus, it is possible to remove an old inner shell in contact with the patient's neck or jaw after use and attach a new inner shell, thereby repeatedly using the outer shell, so that sustainable utilization of resources is possible. In addition, since the cervical collar is adjustably used to have the height corresponding to the neck length of the patient by precisely measuring the neck length, it is possible to provide a correct fit and comfort can be improved when wearing the cervical collar. Further, since the Velcro is locked at a locked portion of an end of the outer shell in a three-point or four-point locking manner, it is possible to efficiently fasten the cervical collar.

### CROSS REFERENCE TO RELATED APPLICATION

The present application claims priority to Korean Patent Application No. 10-2016-0097929, filed Aug. 1, 2016, the entire contents of which is incorporated herein for all purposes by this reference.

### BACKGROUND OF THE INVENTION

### Field of the Invention

The present invention relates generally to a cervical collar. More particularly, the present invention relates to a cervical collar, in which the cervical collar supporting the neck of a patient is separated into an inner shell in contact with the neck of the patient and an outer shell coupled to the inner shell and immobilizing the cervical spine, and the cervical collar is configured such that the inner shell is detachably coupled to the outer shell, and only the inner shell is discarded after use and a new inner shell is coupled to the outer shell, thereby enabling efficient use of resources. The cervical collar having a height corresponding to a patient's neck length is applied to the patient by precisely measuring the neck length of the patient before applying the cervical collar to the patient, thereby providing a correct fit without causing inconvenience of further application and providing comfortable support for the cervical spine of the patient without worsening the neck injury, and Velcro fastening opposite ends of the cervical collar is locked at a locked portion of the outer shell in a three-point or four-point locking manner so that the Velcro can efficiently fasten the cervical collar with one step. Accordingly, the cervical collar is easy to use.

### Description of the Related Art

In general, a cervical collar is widely used at the scene of an accident on an emergency patient or an unconscious patient who has an injury of the cervical spine due to various accidents such as an automobile accident or a fall to protect the cervical spine and to provide safe transport of the patient. In particular, the spinal cord and cervical nerves pass through the spinal canal in the cervical spine, and the motor nerves that travel from the brain to the limbs and the sensory nerves that travel from the limbs and each organ of the body to the brain are connected to the spinal cord and pass through the spinal canal.

In addition, since the autonomic nerves that control the heartbeat, respiration, and digestion pass near the front of the cervical spine, fractures or dislocations of the cervical spine can cause death or limb paralysis. Accordingly, first aid is very important. Thus, a cervical collar is a medical aid that is needed to allow a rescue team to immobilize the cervical spine of the patient at any time in the event of an emergency.

Such a cervical collar is most often carried by rescue team or an emergency rescue team, and such a rescue team or emergency rescue team is equipped with the cervical collar to protect the patient's neck whenever an accident occurs. The most common cervical collar is a mechanical locking device, which is designed to limit head movement. However, since the cervical collar alone cannot provide adequate cervical immobilization, it is used with manual immobilization or other cervical immobilization devices.

However, the cervical collar is supplied at a high price of 30,000 to 70,000 won or more, and the collar is reused, whereby the patient is exposed to a very unsanitary environment because it is merely washed with water rather than discarded. In addition, such conventional cervical collar is configured such that a plate-like support member made of a rigid material and an elastic plate made of an elastic material such as latex foam, which is in contact with the patient's neck or jaw, are coupled to each other by a locking pin. Thus, even though the cervical collar must be discarded after use, it is reused due to a high price thereof and insufficient budget for the rescue team.

Further, in the case of a conventional cervical collar, the neck length of the patient is estimated approximately before applying the collar because the actual neck lengths of patients are different from each other. Accordingly, if the patient feels neck discomfort, the collar is removed and then is replaced with another one, which results in a problem of worsening the cervical spine injury of the patient.

Moreover, in the conventional cervical collar, Velcro that fastens opposite ends of an outer shell of the cervical collar is locked at only one or two points. Thus, when a user removes his/her force after fastening the opposite ends of the cervical collar to each other by interlocking the Velcro, the Velcro locked in only one point or two point locking manner may be stretched by its elastic force. Accordingly, it is required to fasten the loosened Velcro of the cervical collar again, so that the injured cervical spine of the patient, which should be handled with care, may be further injured.

The foregoing is intended merely to aid in the understanding of the background of the present invention, and is not intended to mean that the present invention falls within the purview of the related art that is already known to those skilled in the art.

### SUMMARY OF THE INVENTION

Accordingly, the present invention has been made keeping in mind the above problems occurring in the related art, and an object of the present invention is to provide a cervical collar, in which the cervical collar supporting the neck of a patient is separated into an inner shell in contact with the neck of the patient and an outer shell coupled to the inner shell and immobilizing the cervical spine, and the cervical collar is configured such that the inner shell is detachably coupled to the outer shell, and only the inner shell is discarded after use and a new inner shell is coupled to the outer shell, thereby enabling efficient use of resources.

Another object of the present invention is to provide a cervical collar, in which the cervical collar having a height corresponding to a patient's neck length is applied to the patient by precisely measuring the neck length of the patient before applying the cervical collar, thereby providing a correct fit without causing inconvenience of secondary application, and providing comfortable support for the cervical spine of the patient without worsening the neck injury.

A further object of the present invention is to provide a cervical collar, in which Velcro fastening opposite ends of the cervical collar is locked at a locked portion of the outer shell in a three-point or four-point locking manner so that the Velcro can efficiently fasten the cervical collar with one step. Accordingly, the cervical collar is easy to use.

In order to achieve the above object, according to one aspect of the present invention, there is provided a cervical collar, wherein the cervical collar is made of plastic having a thickness of 0.5 to 2 mm, and includes: an outer shell provided at a front side thereof with a front hole corresponding to an airway of a neck, and at a rear side thereof with a rear hole corresponding to a cervical spine, the outer shell being provided at opposite ends thereof with Velcro and being provided on an inner surface thereof with a plurality of interlocking portions detachably coupled to an inner shell; and the inner shell detachably coupled to the inner surface of the outer shell, and having a plurality of interlocking portions provided at positions corresponding to the interlocking portions of the inner surface of the outer shell and fastened to the interlocking portions of the outer shell, the inner shell being made of a soft material and being in contact with a patient's skin.

In order to achieve the above object, according to another aspect of the present invention, there is provided a cervical collar, wherein the cervical collar is made of plastic having a thickness of 0.5 to 2 mm, and includes: an outer shell provided at a front side thereof with a front hole corresponding to an airway of a neck, and at a rear side thereof with a rear hole corresponding to a cervical spine, the outer shell being provided at opposite ends thereof with Velcro and being coupled with an inner shell at an inner surface thereof, wherein the outer shell is provided at an end thereof with a neck length indicating part including upper and lower portions and accurately measuring a neck length of the patient, so that it is possible to adjust a height of the cervical collar to correspond to the neck length of the patient.

In order to achieve the above object, according to yet another aspect of the present invention, there is provided a cervical collar, wherein the cervical collar is made of plastic having a thickness of 0.5 to 2 mm, and includes: an outer shell provided at a front side thereof with a front hole corresponding to an airway of a neck, and at a rear side thereof with a rear hole corresponding to a cervical spine, the outer shell being provided at opposite ends thereof with Velcro and being coupled with an inner shell at an inner surface thereof, wherein the Velcro fastening opposite ends of the cervical collar is locked at a locked portion of the outer shell in a three-point or four-point locking manner so that the Velcro can efficiently fasten the cervical collar with one step.

According to the present invention, since the cervical collar is capable of detachably separating the outer shell and the inner shell, it is possible to remove an old inner shell in contact with the patient's neck or jaw after use and attach a new inner shell, thereby repeatedly using the outer shell, so that sustainable utilization of resources is possible. In addition, since the cervical collar is adjustably used to have the height corresponding to the neck length of the patient by precisely measuring the neck length, it is possible to provide a correct fit and comfort can be improved when wearing the cervical collar. Further, since the Velcro is locked at the locked portion of the end of the outer shell in the three-point or four-point locking manner, it is possible to efficiently fasten the cervical collar.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a front view showing a cervical collar according to an embodiment of the present invention in a state where an inner shell and an outer shell are fixed to each other;

FIG. 2 is a front view showing the inner shell, an inner surface of the outer shell, and interlocking portions of the cervical collar according to the embodiment of the present invention in a state where the inner shell and the outer shell are separated from each other;

FIGS. 3A, 3B, and 3C are a front view showing a neck length indicating part of the cervical collar according to the embodiment of the present invention, a front view showing an example of an adjusted state, and a front view showing a correct application state for a patient;

FIGS. 4A and 4B are a front view and a rear view showing a fixed state of a front side and a fixed state of a rear side of Velcro of the cervical collar according to the embodiment of the present invention; and

FIG. 5 is a plan view showing a state in which the cervical collar is fastened with the Velcro of the outer shell of the cervical collar according to the embodiment of the present invention.

### DETAILED DESCRIPTION OF THE INVENTION

Hereinbelow, exemplary embodiments of the present invention will be described in detail with reference to the accompanying drawings. Throughout the drawings, the same reference numerals will refer to the same or like parts.

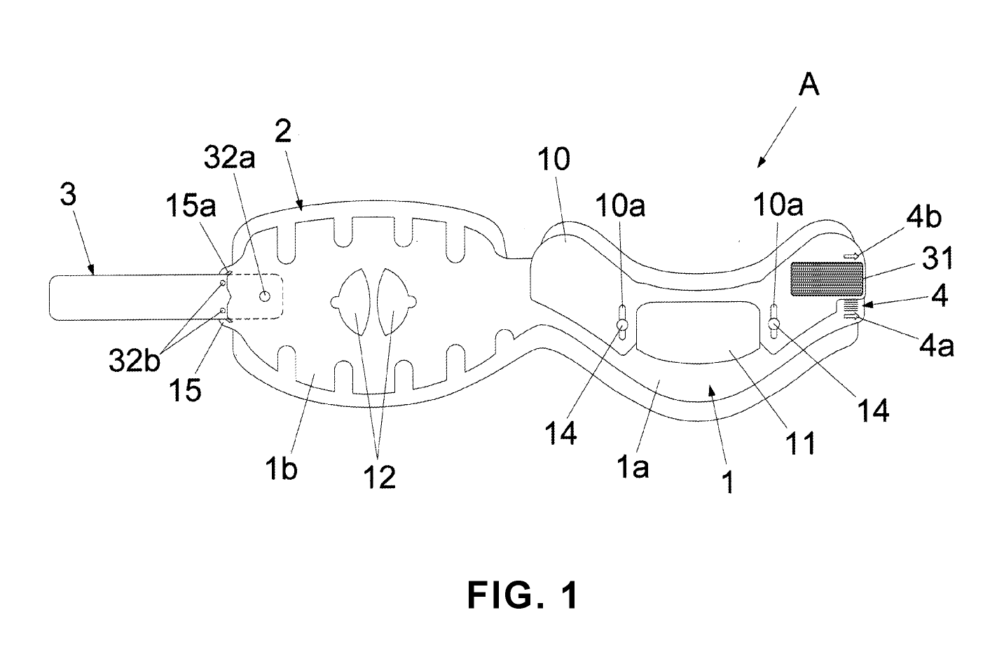

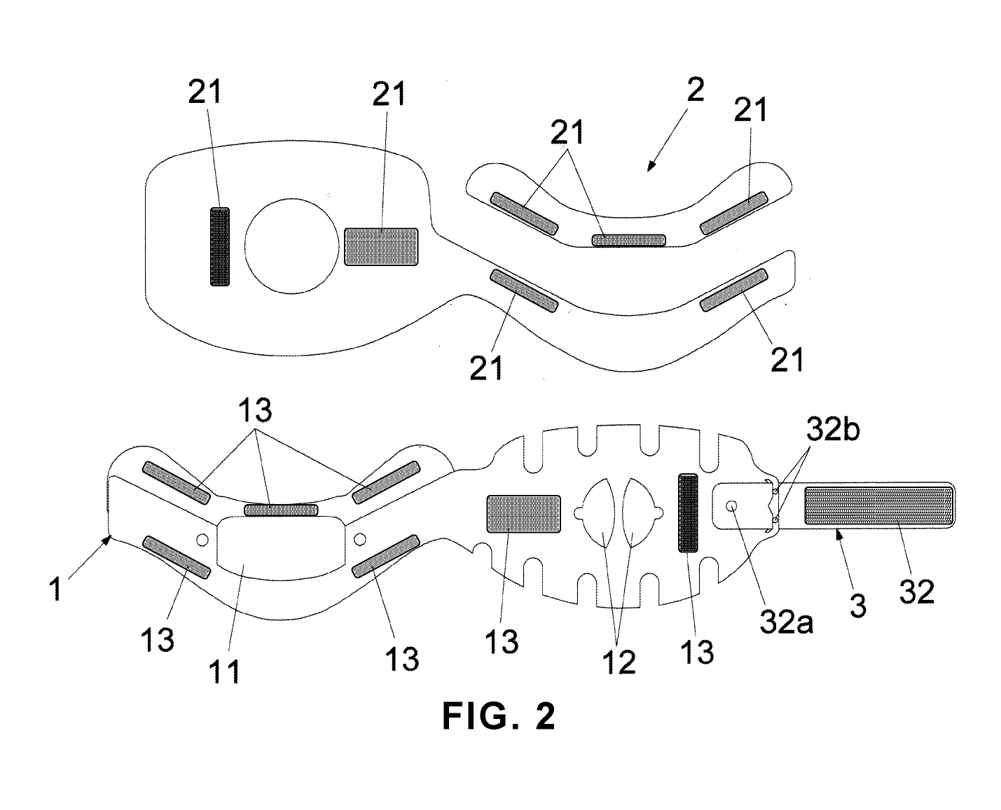

As shown in FIGS. 1 and 2, a cervical collar A according to an embodiment of the present invention is made of plastic having a thickness of 0.5 to 2 mm, and includes: an outer shell 1 provided at a front side thereof with a front hole 11 corresponding to the airway of a neck, and at a rear side thereof with a rear hole 12 corresponding to the cervical spine, the outer shell being provided at opposite ends thereof with Velcro 3, and being provided on an inner surface thereof with a plurality of interlocking portions 13 detachably coupled to an inner shell; and the inner shell 2 detachably coupled to the inner surface of the outer shell 1 and having a plurality of interlocking portions 21 provided at positions corresponding to the interlocking portions 13 of the inner surface of the outer shell 1, and fastened to the interlocking portions 13, the inner shell being made of a soft material and being in contact with a patient's skin.

In this embodiment, the outer shell 1 is made of a plastic material such that opposite ends thereof are not flexible in a vertical direction to support the cervical spine while being flexible in a horizontal direction. However, the outer shell 1 is not limited thereto, and may be made of other materials that are not flexible in the vertical direction while being flexible in the horizontal direction.

The outer shell 1 includes a front outer shell portion 1a supporting a patient's chin and having the front hole 11 formed thereon and a rear outer shell portion 1b supporting the back of a patient's cervical spine and having the rear hole 12 formed thereon. The front outer shell portion 1a includes a movable plate 10 provided at the front outer shell portion 1a to be movable in the vertical direction, and adjusting a height of the collar according to a length of the neck (cervical spine) while supporting the patient's chin. The movable plate 10 is provided with a guide slit 10a extending in the vertical direction at a position adjacent to the front hole 11 so that the movable plate 10 can move in the vertical direction by a movable engagement of a support shaft 14 with the guide slit 10a.

The outer shell 1 is provided on the inner surface thereof with the plurality of interlocking portions 13. In the present embodiment, the interlocking portions 13 use Velcros but are not limited thereto. For example, the interlocking portions 13 may use another structure that performs a detachable interlocking function such as a snap fastener or double-sided tape.

As shown in FIG. 2, seven interlocking portions 13 of the outer shell 1 are provided on inner surfaces of the front and rear outer shell portions 1a and 1b, and seven interlocking portions 21 of the inner shell 2 are provided at positions corresponding to the seven interlocking portions 13 of the outer shell 1. Of course, the number of interlocking portions 13 and 21 may be increased or decreased as required. As such, the outer shell 1 and the inner shell 2 are detachably coupled to each other by the interlocking portions 13 and 21. Thus, after the cervical collar A according to the embodiment of the present invention is put on the patient, the cervical collar A allows the patient to remove an old inner shell 2 from the outer shell 1 by separating the interlocking portions 13 and 21 from each other and to attach a new inner shell 2 to the outer shell 1 by interlocking the portions 13 and 21. Thus, the outer shell 1 can be reused, so that the cervical collar A according to the embodiment of the present invention can be worn in a clean state on the patient.

The front outer shell portion 1a is provided at an end thereof with a neck length indicating part 4 measuring and indicating the neck length of the patient.

The neck length indicating part 4 includes a lower indication portion 4a provided at a lower part of the front outer shell portion 1a and an upper indication portion 4b provided at an upper part of the movable plate 10 moving on the front outer shell portion 1a. Here, a graduation is provided between the upper and lower indication portions 4a and 4b, so that it is possible to conveniently measure the neck length of the patient.

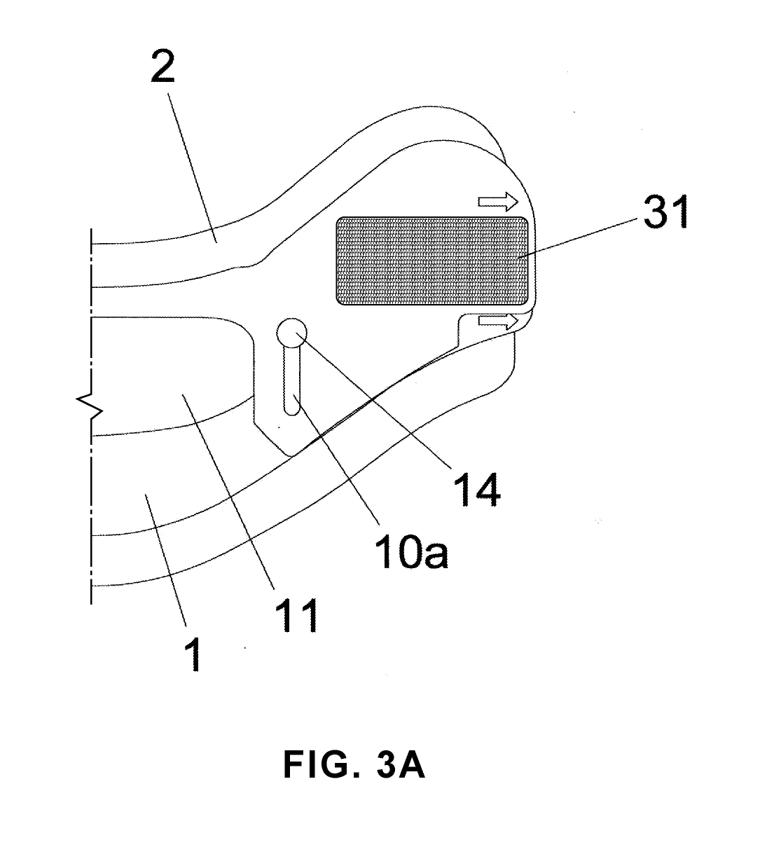

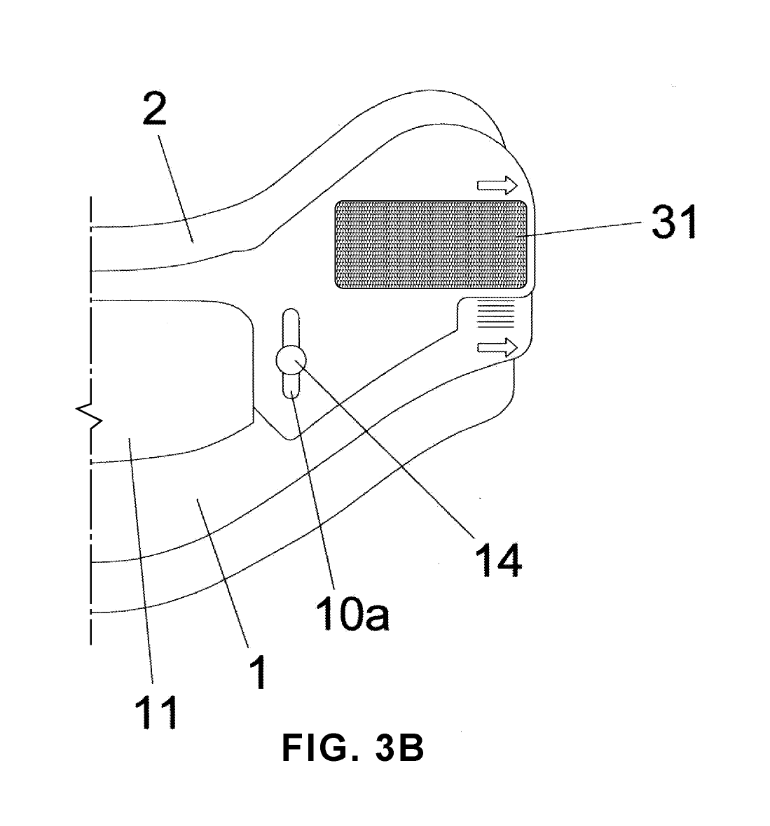

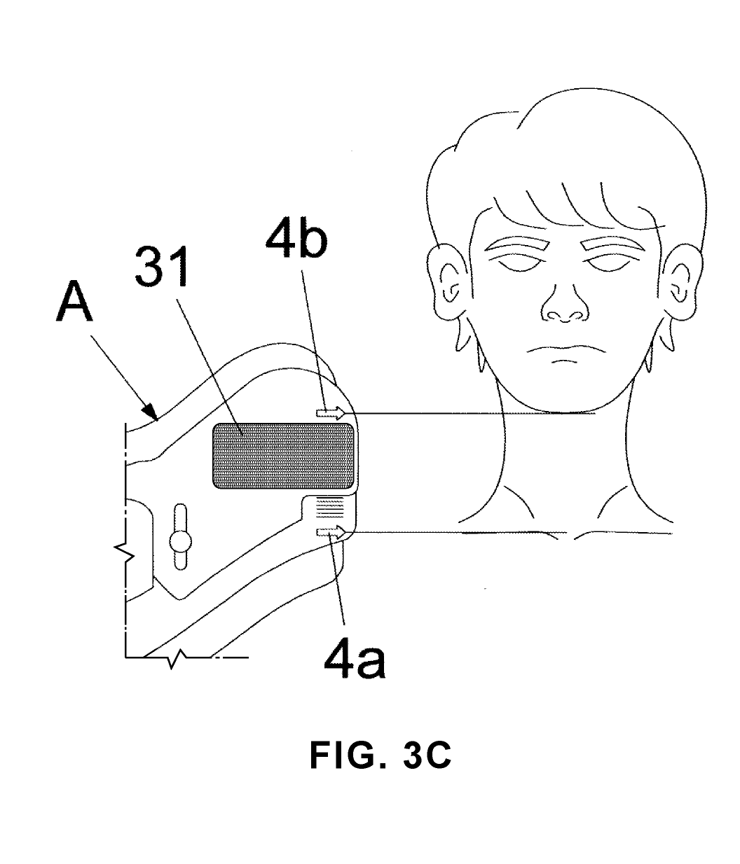

Referring to FIG. 3C, first, the neck length indicating part 4 approaches the cervical spine of the patient and then a length between the patient's chin (lifted state is preferable) and a mid-point of the collar bone (near the clavicle) is measured by lifting the movable plate 10 to obtain a precise length, thereby efficiently measuring the neck length of the patient. Thus, the cervical spine of the patient can be enclosed and immobilized in the state where the neck length of the patient is precisely measured, so that it is possible to comfortably immobilize the cervical spine of the patient in a precise position.

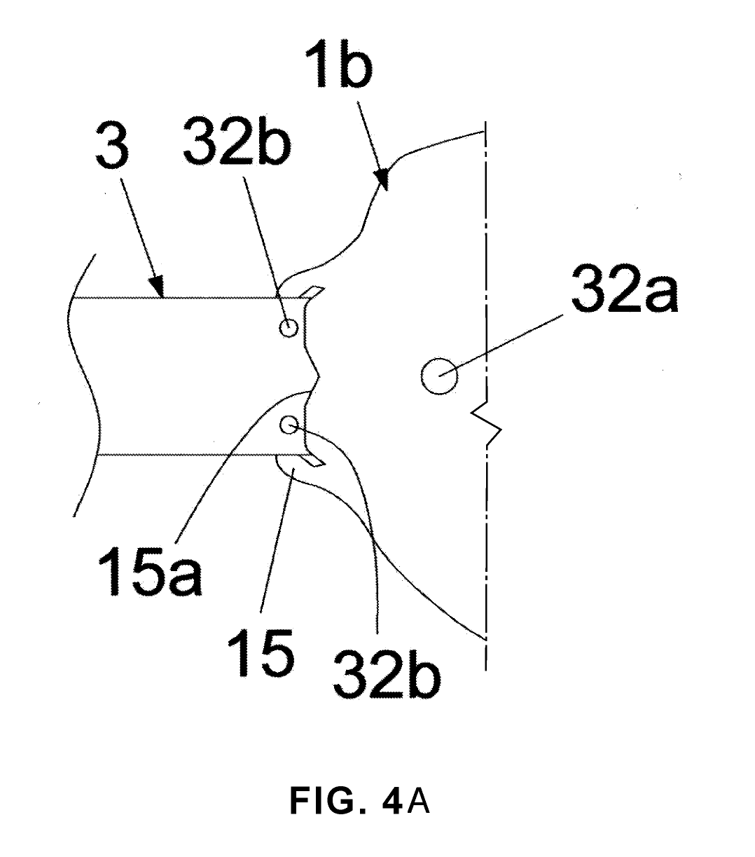

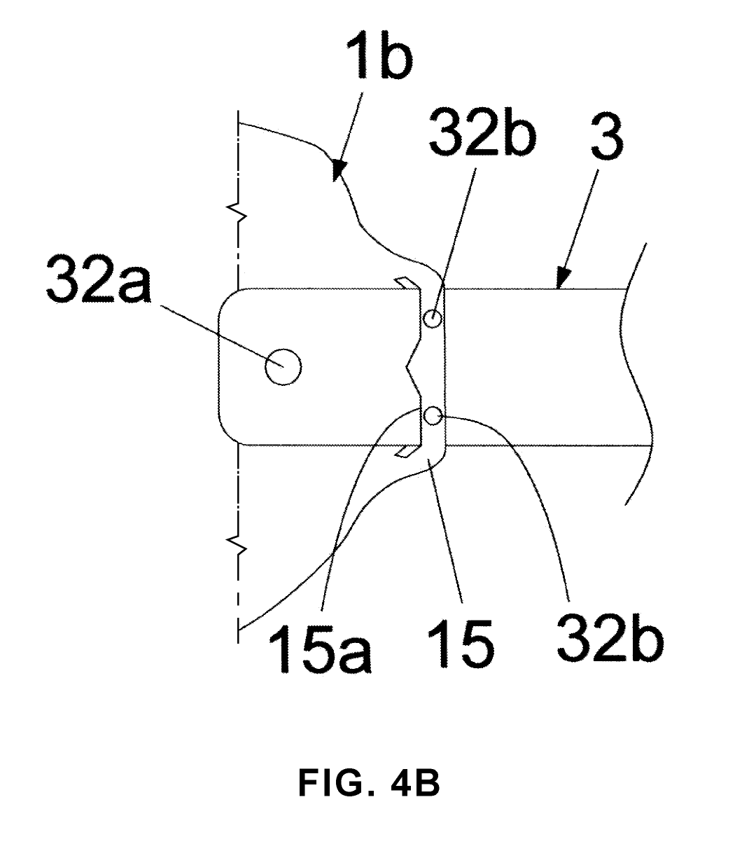

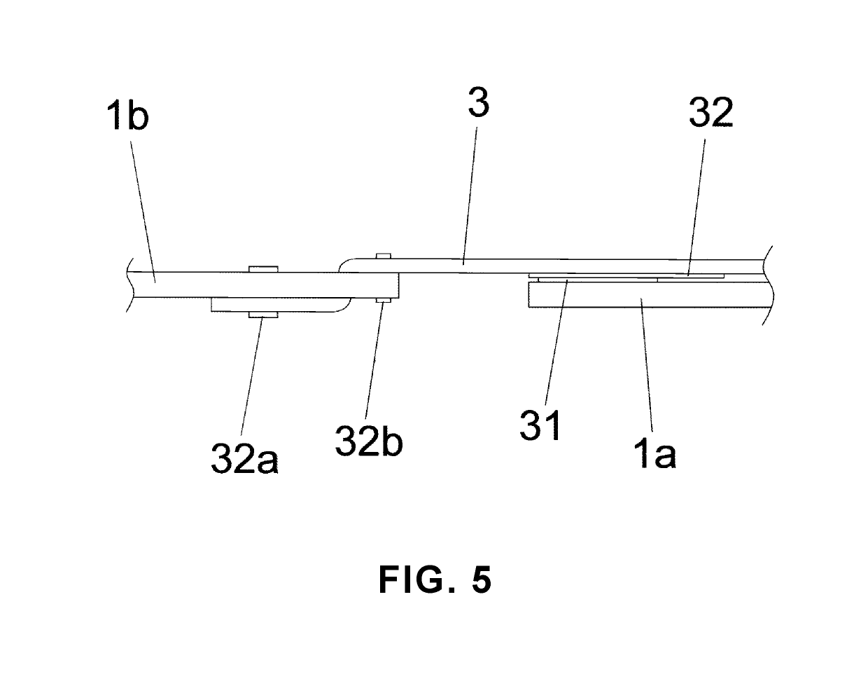

Further, as shown in FIG. 1, and FIGS. 4A and 4B, the cervical collar A according to the embodiment of the present invention includes a hook part 31 of Velcro provided at the end of the front outer shell portion 1a, and a pile part 32 of Velcro provided at an end of the rear outer shell portion 1b. The pile part 32 of Velcro is locked to the rear outer shell portion 1b such that an end of the pile part 32 of Velcro is locked to the end of the rear outer shell portion 1b by a locking piece 32a, and a Velcro locking piece 32b locking the pile part 32 of Velcro and a locking rib 15 integrally with each other is fixed to the locking rib 15, the locking rib 15 being provided with a through hole 15a through which the pile part 32 of Velcro provided at the rear outer shell portion 1b passes. Accordingly, the pile part 32 of Velcro is locked by both the locking piece 32a and the Velcro locking piece 32b so as not to be movable. In other words, as shown in FIG. 5, when fastening the Velcro 3, a locked portion of the pile part 32 of Velcro is not changed in length during interlocking of the hook part 31 of Velcro and the pile part 32 of Velcro, the fastening of the Velcro can be performed safely. Thus, the cervical spine of the patient can be immobilized comfortably without secondary repetitive fastening.

The cervical collar according to the present invention can be said to be an industrially applicable invention since it is possible to produce the same product repeatedly in a cervical collar manufacturing industry manufacturing a general cervical collar.

While the foregoing illustrates and describes the present invention using certain preferred embodiments as examples, the present invention is not limited to the embodiments disclosed above. Various modifications and alterations can be made by those having ordinary skill in the field of art to which the present invention pertains without departing from the spirit of the present invention.

What is claimed is:

1. A cervical collar, wherein the cervical collar (A) is made of plastic having a thickness of 0.5 to 2 mm, and comprises:
an outer shell (1) provided at a front side thereof with a front hole (11) corresponding to an airway of a neck, and at a rear side thereof with a rear hole (12) corresponding to a cervical spine, the outer shell being provided at opposite ends thereof with Velcro (3) or a snap fastener and being provided on an inner surface thereof with a plurality of interlocking portions (13) detachably coupled to an inner shell; and
the inner shell (2) detachably coupled to the inner surface of the outer shell (1), and having a plurality of interlocking portions (21) provided at positions corresponding to the interlocking portions (13) of the inner surface of the outer shell and fastened to the interlocking portions (13) of the outer shell, the inner shell being made of a soft material and being in contact with a patient's skin,
wherein the outer shell (1) is provided at the inner surface thereof with the plurality of interlocking portions (13),
the plurality of interlocking portions (13) of the outer shell (1) are provided on an inner surface of a front outer shell portion (1a) and on an inner surface of a rear outer shell portion (1b), and
the plurality of interlocking portions (21) of the inner shell (2) are provided at the positions corresponding to the interlocking portions (13) of the outer shell, so that due to the interlocking portions (13 and 21), an old inner shell (2) is removed from the outer shell (1) after use and a new inner shell (2) is coupled to the outer shell.

2. A cervical collar, wherein the cervical collar (A) is made of plastic having a thickness of 0.5 to 2 mm, and comprises:
an outer shell (1) provided at a front side thereof with a front hole (11) corresponding to an airway of a neck, and at a rear side thereof with a rear hole (12) corresponding to a cervical spine, the outer shell being provided at opposite ends thereof with Velcro (3) and being coupled to an inner shell on an inner surface thereof; and
the inner shell (2) coupled to the outer shell (1),
wherein the outer shell (1) includes: a front outer shell portion (1a) supporting a chin of a patient and having a front hole (11); and a rear outer shell portion (1b) supporting a back of a cervical spine of the patient and having a rear hole (12),
wherein the front outer shell portion (1a) includes a movable plate (10) provided at the front outer shell portion (1a) to be movable in a vertical direction, and adjusting a height of the collar according to a length of a neck (cervical spine) of the patient while supporting the chin of the patient, the movable plate being provided with a guide slit (10a) extending in the vertical direction at a position adjacent to the front hole (11) so that the movable plate moves in the vertical direction by a movable engagement of a support shaft (14) with the guide slit (10a), and
the front outer shell portion (1a) is provided at an end thereof with a neck length indicating part (4) measuring and indicating a neck length of the patient,
the neck length indicating part (4) including a lower indication portion (4a) provided at a lower part of the front outer shell portion (1a) and an upper indication portion (4b) provided at an upper part of the movable plate (10) moving on the front outer shell portion (1a), with a graduation provided between the upper and lower indication portions (4a) and (4b).

3. A cervical collar, wherein the cervical collar (A) is made of plastic having a thickness of 0.5 to 2 mm, and comprises:
an outer shell (1) provided at a front side thereof with a front hole (11) corresponding to an airway of a neck, and at a rear side thereof with a rear hole (12) corresponding to a cervical spine, the outer shell being provided at opposite ends thereof with Velcro (3) and being coupled to an inner shell on an inner surface thereof; and
the inner shell (2) coupled to the outer shell (1),
wherein the outer shell (1) is provided with a hook part (31) of Velcro provided at an end of a front outer shell portion (1a) of the outer shell, and a pile part (32) of Velcro provided at an end of a rear outer shell portion (1b) of the outer shell,
wherein the pile part (32) of Velcro provided at the end of the rear outer shell portion (1b) is locked at an end thereof to the rear outer shell portion (1b) by a locking piece (32a), and a Velcro locking piece (32b) locking the pile part (32) of Velcro and a locking rib (15) integrally with each other is fixed to the locking rib (15), the locking rib (15) being provided with a through hole (15a) through which the pile part (32) of Velcro provided at the rear outer shell portion (1b) passes.
